(19) United States
(12) Patent Application Publication
(10) Pub. No.: US 2022/0265342 A1
(43) Pub. Date: Aug. 25, 2022

(54) TREATMENT DEVICE WITH DAMPING FEATURE

(71) Applicant: OLYMPUS MEDICAL SYSTEMS CORP., Tokyo (JP)

(72) Inventors: Hiroshi ASHIBA, Tokorozawa-shi (JP); Yasuhiro MAEDA, Tokyo (JP); Minoru KATSUMATA, Tokyo (JP)

(73) Assignee: OLYMPUS MEDICAL SYSTEMS CORP., Tokyo (JP)

(21) Appl. No.: 17/667,592

(22) Filed: Feb. 9, 2022

Related U.S. Application Data
(60) Provisional application No. 63/152,888, filed on Feb. 24, 2021.

(51) Int. Cl. A61B 18/14

(52) U.S. Cl. CPC A61B 18/1445; A61B 2018/00994

(57) ABSTRACT

Treatment device for ultrasonic treatment and high frequency treatment procedure is equipped with an ultrasonic transducer including piezoelectric elements converting electrical power into ultrasonic vibrations. The treatment device includes a transmission rod with a treatment probe and jaw for clasping objects. The transmission rod includes features for damping, such as a sheath, a geometry of the outer surface of the transmission rod, or combinations of such features, to minimize or prevent excess vibrations and to, among other things, decrease frictional heat caused by the friction between the damping features and the transmission rod arising from attenuating the ultrasonic vibrations.

### RELATED APPLICATION DATA

This application is based on and claims priority under 35 U.S.C. § 119 to U.S. Provisional Application No. 63/152,888 filed on Feb. 24, 2021, the entire contents of which are incorporated herein by reference.

### FIELD OF DISCLOSURE

The present invention relates to an ultrasonic treatment device used for dissecting and coagulating tissues. The ultrasonic treatment device is equipped with an ultrasonic transducer including piezoelectric elements converting electrical power into ultrasonic vibrations. The ultrasonic vibrations are transmitted along the transmission member to a probe that serves to clasp objects together with a jaw. The transmission member may create undesired transverse vibration that causes problems such as deterioration of blood vessel sealing performance, heat generation, abnormal stress, and abnormal noise.

### BACKGROUND

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art against the present invention.

FIG. 9 is a figure of an ultrasonic treatment device in the related art (U.S. Pat. No. 8,696,666). The related art surgical operation system 1 consists of a handpiece 2, a main body apparatus 3 which is an output control apparatus, a foot switch 4 and a counter electrode plate 5. The handpiece 2 is a surgical treatment instrument capable of treatment using both ultrasonic and high-frequency current. The handpiece 2 is connected to the main body apparatus 3 via a cable 2 a which is attachable and detachable. The handpiece 2 has an insertion portion 2b and a handle portion 2c. The connector portion 3a connects the handpiece to the main body apparatus 3, which controls the output of the ultrasonic vibration and/or high-frequency current. The main body apparatus 3 has a plurality of displays 3b and a plurality of various operation buttons 3c for controlling the performance of handpiece 2. The foot switch 4 is connected to the main body apparatus 3 through a cable 4 a, and switches the mode from treatment using ultrasonic vibration, treatment using high-frequency current, or treatment using both. The counter electrode plate 5 is connected to the main body apparatus 3 through a cable 5 a. The counter electrode plate 5 is a return electrode for returning a current which passes through a subject at the time of monopolar output of a high-frequency current.

FIG. 10 is a figure of a portion of an ultrasonic treatment device in the related art (U.S. Pat. No. 5,989,275). The related art ultrasonic treatment device includes a transmission rod 86 used for transmitting ultrasonic vibrations to the ultrasonic probe. The transmission rod 86 is covered by a damping sheath 160, which is further covered by the elongated tubular member 174. Diametrically opposed openings 162b and 162c, as well as longitudinal slit 164 are formed on the damping sheath 160. Compliant members 190b and 190c (O-rings and fenders) are disposed around the periphery of the damping sheath 160, which are preferably disposed around the nodes to minimize damping of the desired longitudinal vibration.

The damping sheath 160 is constructed of a polymeric material, preferably with a low coefficient of friction to minimize dissipation of energy from the axial motion or longitudinal vibration of the transmission rod 86. The damping sheath 160 is preferably in light contact with the transmission rod 86 to dampen or limit non-axial or transverse side-to-side vibration of the transmission rod 86. The damping sheath 160 can dampen transverse motion occurring near multiple nodes and antinodes of the unwanted vibration which are located randomly along the length of the transmission rod 86 relative to the nodes and antinodes of the desired longitudinal vibration.

Transverse vibrations occurring in ultrasonic treatment devices when the ultrasonic probe is vibrated can lead to problems, such as deterioration of blood vessel sealing performance, heat generation, abnormal stress, and abnormal noise. Even though previous ultrasonic treatment devices may have structures, such as the damping sheath 160, such a damping sheath 160 is in contact throughout the transmission rod 86 in areas where dampening or limiting the non-axial or transverse side-to-side vibration is not necessary. Additionally, this configuration may cause problems such as heat generation through friction between the transmission rod 86 and the damping sheath 160 due to longitudinal vibration.

### SUMMARY

Accordingly, there is a need for designing an ultrasonic treatment device with an efficient structure in view of the practical usage, which would substantially obviate one or more of the issues due to limitations and disadvantages of related art treatment devices. An object of the present disclosure is to provide an improved treatment device having an efficient structure and practical administration of the associated medical procedure. For example, there is a need to provide improved damping solutions that, for example, minimize the contact between a transmission rod and a damping structure, such as a sheath, so as to minimize or prevent heat generation or other issues to arise. At least one or some of the objectives is achieved by the treatment device disclosed herein.

Additional features and advantages will be set forth in the description that follows, and in part will be apparent from the description, or may be learned by practice of the invention. The objectives and other advantages of the disclosed treatment device will be realized and attained by the structure particularly pointed out in the written description and claims thereof, as well as the appended drawings.

In general, the disclosed structures and systems provide for an ultrasonic treatment device efficiently suppressing problems such as deterioration of blood vessel sealing performance, heat generation, abnormal stress, and abnormal noise created from vertical and/or horizontal ultrasonic vibrations.

Embodiments of the disclosed surgical treatment device comprises a transducer generating ultrasonic vibrations, a transmission rod including a treatment probe in which a proximal end of the transmission rod is operatively connected to the transducer for transmitting ultrasonic vibration generated by the transducer to the treatment probe located at the distal end, and a damping feature for attenuating vibrations movably placed along the longitudinal axis direction of the transmission rod between an adjacent node and antinode of the longitudinal vibration. The transmission rod includes a tapered portion having an outer surface that is inclined inwardly from a distal end of the tapered portion towards a proximal end of the tapered portion and the outer surface of the tapered portion has a maximum outer diameter that is larger than an inner diameter of the damping feature. Furthermore, along the transmission rod, the node of the longitudinal vibration is located more distally than the tapered portion of the transmission rod.

In some embodiments, a surgical treatment device comprises a transducer generating ultrasonic vibrations, a transmission rod including a treatment probe in which a proximal end of the transmission rod is operatively connected to the transducer for transmitting ultrasonic vibration generated by the transducer to the treatment probe located at the distal end, and a damping feature for attenuating vibrations movably placed along the longitudinal axis direction of the transmission rod between an adjacent node and antinode of the longitudinal vibration. The transmission rod includes a tapered portion having an outer surface that is inclined inwardly from the proximal end of the tapered portion towards the distal end of the tapered portion and the outer surface of the tapered portion has a maximum outer diameter that is larger than an inner diameter of the damping feature. Furthermore, along the transmission rod, the node of the longitudinal vibration is located more proximally than the tapered portion of the transmission rod.

In some embodiments, the damping feature is a tube.

In some embodiments, the damping feature is a sleeve.

In some embodiments, the damping feature includes a tapered portion.

In some embodiments, the damping feature includes a slit.

In some embodiments, a rubber ring is placed at the node.

In some embodiments, the transmission rod includes a first portion having an outer diameter larger than the inner diameter of the damping feature, wherein, along the transmission rod, the first portion is placed more proximally than the tapered portion of the transmission rod.

In some embodiments, the transmission rod includes a first portion having an outer diameter larger than the inner diameter of the damping feature, wherein, along the transmission rod, the first portion is placed more distally than the tapered portion of the transmission rod.

In some embodiments, the first portion is located proximate to the antinode of the longitudinal vibration.

In some embodiments, the first portion is removable from the transmission rod.

In some embodiments, the first portion is assembled together with the transmission rod through a screw.

In some embodiments, the first portion is assembled together with the transmission rod through shrink fitting.

In some embodiments, the treatment probe is configured to treat living tissue.

In some embodiments, the treatment probe is configured as an electrode for treatment using high frequency currents.

In some embodiments, the treatment probe includes a curved shape.

In some embodiments, a transmission rod comprises an elongate body configured for transmitting ultrasonic vibration from a proximal end to a distal end and a treatment probe formed at the distal end of the elongate body. The treatment probe includes a treatment surface and a tapered portion having an outer surface that is inclined inwardly from a distal end of the tapered portion towards a proximal end of the tapered portion, and along the transmission rod, a node of the ultrasonic vibration is located more distally than the tapered portion of the transmission rod.

In some embodiments, a transmission rod comprises an elongate body configured for transmitting ultrasonic vibration from a proximal end to a distal end and a treatment probe formed at the distal end of the elongate body, wherein the treatment probe includes a treatment surface. The transmission rod includes a tapered portion having an outer surface that is inclined inwardly from a proximal end of the tapered portion towards a distal end of the tapered portion, and along the transmission rod, a node of the ultrasonic vibration is located more proximally than the tapered portion of the transmission rod.

In some embodiments, the treatment probe includes a jaw moveable relative to the treatment surface from an open position to a closed position.

In some embodiments, a rubber ring is placed at a node of the ultrasonic vibrations.

In some embodiments, the transmission rod includes a first portion having an outer diameter larger than the other portions of the transmission rod.

In some embodiments, the first portion is placed more distally than the tapered portion of the transmission rod.

In some embodiments, the first portion is placed more proximally than the tapered portion of the transmission rod.

In some embodiments, the first portion is located at an antinode of the longitudinal vibration.

In some embodiments, the first portion is removable from the transmission rod.

In some embodiments, the first portion may be assembled together with the transmission rod with a screw.

In some embodiments, the thickened portion may be assembled together with the transmission rod with a shrink fitting.

In some embodiments, the transmission rod is configured as an electrode for treatment using high frequency currents.

In some embodiments, the treatment probe is configured to treat living tissues.

In some embodiments, the treatment probe is configured as an electrode for treatment using high frequency currents.

In some embodiments, the treatment probe has a curved shape.

Other systems, methods, features and advantages will be, or will become, apparent to one with skill in the art upon examination of the following figures and detailed description. It is intended that all such additional systems, methods, features and advantages be included within this description, be within the scope of the present disclosure, and be protected by the following claims. Nothing in this section should be taken as a limitation on those claims. Further aspects and advantages are discussed below in conjunction with the embodiments of the disclosed input device. It is to be understood that both the foregoing general description and the following detailed description of the disclosed input device are examples and explanatory and are intended to provide further explanation of the disclosed input device as claimed.

### BRIEF DESCRIPTION OF THE DRAWING

The following detailed description of preferred embodiments can be read in connection with the accompanying drawings in which like numerals designate like elements and in which:

FIG. 1 shows an embodiment of a treatment device.

FIG. 2 shows a magnified view of the treatment end of the treatment device in Area P in FIG. 1.

FIG. 3A is a top view of a treatment region of an ultrasonic probe and FIG. 3B is an exaggerated representation, based on a simulation, of the ultrasonic vibrations of the treatment region in transverse vibration mode.

FIG. 4A is a side view of a treatment region of an ultrasonic probe and FIG. 4B is an exaggerated representation, based on a simulation, of the ultrasonic vibrations of the treatment region in transverse vibration mode.

FIG. 5 is an exaggerated perspective view of a treatment region of an ultrasonic probe and showing the variation in transverse vibration during vibration of the ultrasonic probe.

FIG. 6 illustrates a damping structure and associated features of the transmission member of an ultrasonic probe.

FIG. 7 illustrates a damping structure and associated features of the transmission member of an ultrasonic probe according to an embodiment where the damping structure is located at a probe axial position proximate the antinode position of the transverse vibration

FIGS. 8A to 8C illustrate variations in the disclosed damping structure.

FIG. 9 shows an ultrasonic treatment device in the related art.

FIG. 10 shows a portion of an ultrasonic treatment device in the related art.

Throughout all of the drawings, dimensions of respective constituent elements are appropriately adjusted for clarity. For ease of viewing, in some instances only some of the named features in the figures are labeled with reference numerals.

### DETAILED DESCRIPTION

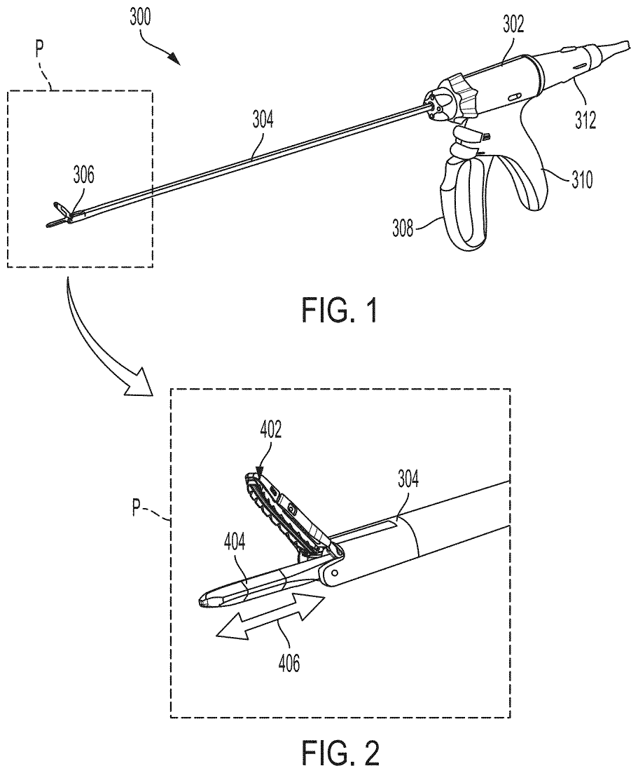

FIG. 1 is an illustration of a surgical treatment device 300 including a body 302, a sheath 304, and a treatment end 306. The body 302 includes a moving arm 308, a grip 310, and a transducer 312. The moving arm 308 is used together with grip 310 to actuate and operate the functions of treatment end 306. The transducer 312 includes an ultrasonic transducer which is connected to a power source supplying power used for ultrasonic treatment, as well as high-frequency treatment of surgical treatment device 300. The power source can be a wired or wireless power source. The sheath 304 protects the wires and transmission members within, necessary for operating the functions of treatment end 306.

FIG. 2 is the magnified illustration of the treatment end 306 of the surgical treatment device 300. The treatment end 306 consists of a jaw 402 and an ultrasonic probe 404. In the current embodiment, the jaw 402 and the ultrasonic probe 404 open and close in the vertical direction through the manipulation of the movable handle 308 in order to grasp tissues and other objects for treatment, but ultrasonic probe 404 may be used for the treatment procedures without a jaw. The ultrasonic probe 404 vibrates at an ultrasonic frequency transmitted through the transmission member within sheath 304. A longitudinal vibration, an ultrasonic vibration of the ultrasonic probe 404 made in the direction 406, creates frictional heat used for treatment purposes such as dissection of tissues, as well as frictional heat caused through contacting objects such as damping members. The ultrasonic probe 404 may have a curved shape and may also serves as an electrode for treatment using high frequency currents.

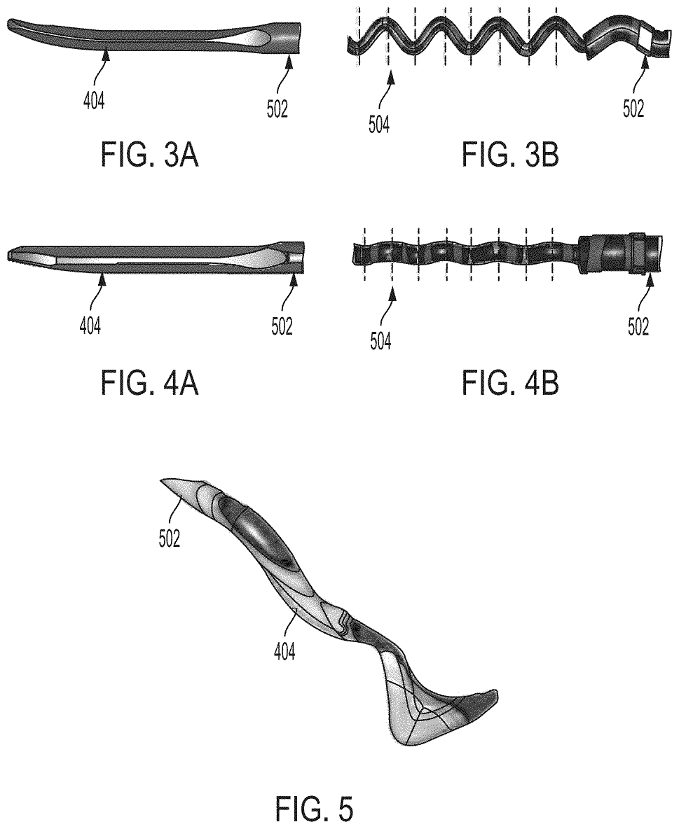

FIG. 3A illustrates the ultrasonic probe 404 viewed from the vertical direction, the direction the jaw 402 opens and closes. FIG. 3A also illustrates the transmission member 502 extending from the ultrasonic probe 404 on the distal end, extending within the sheath 304, and connecting to the transducer 312 at the proximal end. The ultrasonic probe 404 and transmission member 502 are in its stationary state, a state where neither the ultrasonic vibration nor the high frequency current is applied to the ultrasonic probe 404 and transmission member 502.

FIG. 3B also illustrates the ultrasonic probe 404 viewed from the vertical direction, the direction the jaw 402 opens and closes. FIG. 3B illustrates an exaggerated representation of the ultrasonic probe 404 and transmission member 502 in its oscillated state, a state where the ultrasonic vibration is applied.

Considering the use of ultrasonic probe 404 in treatment procedures, longitudinal vibration would be the desirable ultrasonic vibration. On the contrary, transverse vibrations and torsional vibrations would be undesirable ultrasonic vibrations that may cause issues during the treatment procedures. Because the ultrasonic probe 404 is curved in the horizontal direction with an aim to improve the visibility during the treatment procedure, the axial unbalance of the ultrasonic probe 404 in the horizontal direction may create substantial transverse vibrations when the ultrasonic vibration is applied to the ultrasonic probe 404. In the case shown in FIG. 3B, the ultrasonic vibration has caused a strong transverse vibration at the antinode 504 of the transverse vibration leading to problems such as deterioration of blood vessel sealing performance, heat generation, abnormal stress, and abnormal noise.

FIG. 4A illustrates the ultrasonic probe 404 viewed from the horizontal direction, the direction perpendicular to the vertical direction referred to in FIGS. 3A and 3B. FIG. 4A also illustrates the transmission member 502 extending from the ultrasonic probe 404, extending within the sheath 304, and connecting to the transducer 312. The ultrasonic probe 404 and transmission member 502 are in its stationary state, a state where neither the ultrasonic vibration nor the high frequency current is applied to the ultrasonic probe 404 and transmission member 502.

FIG. 4B also illustrates the ultrasonic probe 404 viewed from the horizontal direction. FIG. 4B illustrates an exaggerated representation of the ultrasonic probe 404 and the transmission member 502 in its oscillated state, a state where the ultrasonic vibration is applied. Because the ultrasonic probe 404 is not curved in the vertical direction, axial unbalance in the vertical direction is minimal compared to the axial unbalance due to the curved ultrasonic probe 404 curving in the horizontal direction. Thus, the undesired transverse vibrations that may occur at the antinode 504 at the time of application of ultrasonic vibration is weak compared to the transverse vibrations in the horizontal direction as disclosed in FIG. 3B.

FIG. 5 also illustrates an exaggerated representation of the ultrasonic probe 404 and the transmission member 502 in its perspective view. FIG. 5 illustrates the ultrasonic probe 404 and transmission member 502 in its oscillated state, showing the occurrence of undesired transverse vibration created due to the curve of the ultrasonic probe 404.

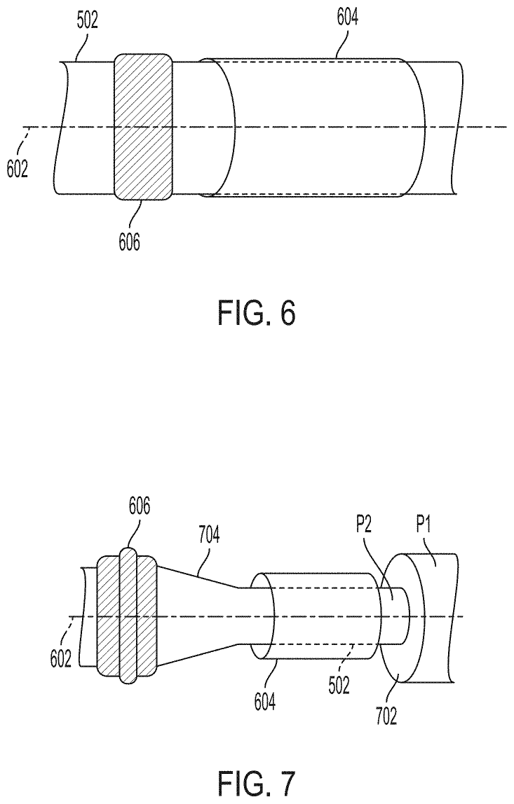

FIG. 6 illustrates the transmission member 502, extending in the direction of center axis 602, covered by a damping feature, such as an attenuation tube 604. The longitudinal vibration occurs in parallel to the center axis 602 and the undesired transverse vibration occurs in the direction perpendicular to the center axis 602 and the longitudinal vibration. The attenuation tube 604 comes in contact with the transmission member 502 or the rubber member 606 and serves to attenuate the transverse vibrations caused by the ultrasonic vibration applied to the transmission member 502. In order to suppress the frictional heat caused by the longitudinal vibration and contact between the attenuation tube 604 and the transmission member 502, it is preferred to place the attenuation tube 604 at the node or near the node of the longitudinal vibration. In order to attenuate the transverse vibration caused by the ultrasonic vibration applied to the transmission member 502, it is preferred to place the attenuation tube 604 at a location including at least one antinode of the transverse vibration. The attenuation tube 604 is made from polymer materials such as fluororesins, PTFE, FEP, and PFA with a thickness around 0.1 to 1.0 mm. The attenuation tube 608 may include a linear or helical slit for easing the attachment to the transmission member 502. Attenuation tube 604 may consist a sleeve structure.

Although the structure of the attenuation tube 604 in FIG. 6 may attenuate transverse vibrations occurring in ultrasonic treatment devices when the ultrasonic probe is vibrated, because of the large amount of surface area contact between the attenuation tube 604 and the outer surface of the transmission member 502, problems such as heat generation through friction between the transmission member 502 and the attenuation tube 604 through longitudinal vibration may occur. Also, the inner diameter of the attenuation tube 604 may widen through usage, which can over time reduce the contacting area between the transmission member 502 and the attenuation tube 604 and problems associated with transverse vibrations of the ultrasonic vibrations can occur, such as deterioration of blood vessel sealing performance, heat generation, abnormal stress, and abnormal noise.

FIG. 7 illustrates an embodiment of a transmission member 502 having a tapered structure in combination with attenuation tube 604. The attenuation tube 604 is placed between the step structure 702 that is formed by a portion P1 of the transmission member 502 having a larger outer diameter compared to a portion P2 of the transmission member 502, and a node structure 606 at a node of the longitudinal vibration of the transmission member 502. The portion P2 of the transmission member 502 can have an outer surface with a constant diameter along the longitudinal length, i.e., in a direction along center axis 602. Alternatively, and as shown in FIG. 7, the portion P2 of the transmission member 502 can have an outer surface with at least a portion having a changing diameter along the longitudinal length, preferably a diameter that has a constant rate of change as a function of position along the longitudinal length. In some embodiments, the portion of the outer surface having a changing diameter forms a conical geometry or is tapered section 704. It should be noted that the size of the inner diameter surface of the attenuation tube 604 is larger than the diameter of the outer surface of portion P2 where P2 meets the step structure 702; but the size of the inner diameter surface of the attenuation tube 604 is less than the diameter of the outer surface at least a portion of the tapered section 704. The longitudinal distance of portion P2 separating the step structure 702 from the location on the tapered section 704 where the diameter of the outer surface is equal to or is larger than the inner diameter surface of the attenuation tube 604 defines a length of the transmission member 502 along which the attenuation tube 604 can move.

Step structure 702 may be made removable from transmission member 502 by, e.g., separation of portion P1 from portion P2, in order to ease the assembly of the attenuation tube 604 and transmission member 502. For example, the step structure 702 may be assembled together with the transmission member 502 through a screw or shrink fitting the large dimeter portion P1 onto smaller diameter portion P2. Since the attenuation tube 604 is slidably located between the step structure 702 and tapered portion 704 (as described above), the attenuation tube 604 has the tendency to move towards the node position of longitudinal vibration (such as at 47 kHz or 55 kHz) during oscillation of the transmission member 502, such as during operation of a treatment device incorporating the transmission member 502. Also, it is preferred to set the antinode position of the longitudinal vibration of the ultrasonic vibration near the step structure 702 and the node position of the longitudinal vibration of the ultrasonic vibration near the node structure 606, such as at rubber member shown in FIGS. 6, 7 and 8A to 8C. Through this configuration, the attenuation tube 604 would move towards the node structure 606 and would be stopped at the tapered section 704 of the transmission member 502 where the outer diameter of the tapered section 704 equals the inner diameter of the attenuation tube 604. Even if, with use, the inner diameter of the attenuation tube 604 becomes larger than the tapered section 704, the attenuation tube 604 would be stopped at the node structure 606 by the rubber member.

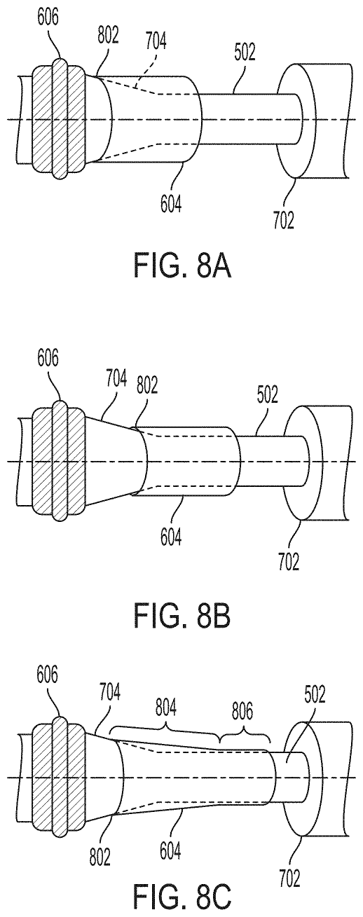

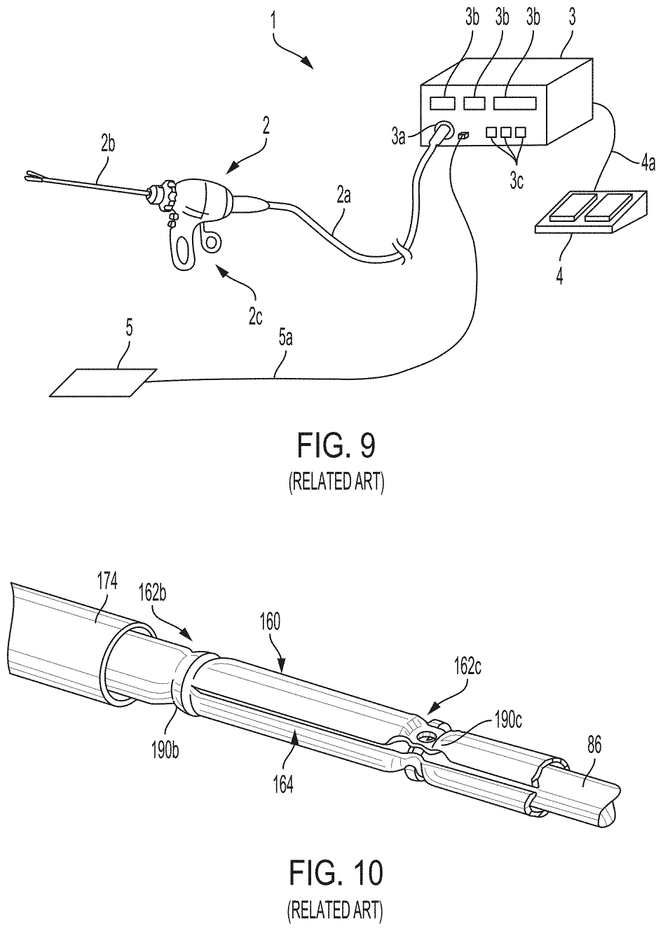

FIGS. 8A to 8C illustrate variations in the disclosed damping structure. FIG. 8A illustrates the transmission member 502 having a tapered structure in combination with attenuation tube 604, where the transmission member 502 and attenuation tube 604 is in an oscillated state. The attenuation tube 604 placed between the step structure 702 and tapered section 704 is moved towards the node position located near the node structure 606 through the longitudinal vibration (such as at 47 kHz or 55 kHz) during oscillation. The attenuation tube 604 is stopped at the contacting diameter 802 where the outer diameter of the tapered section 704 equals the inner diameter of the attenuation tube 604. Because the contact between the transmission member 502 and the attenuation tube 604 is minimized to be at the contact diameter 802, the frictional heat created between the transmission member 502 and the attenuation tube 604 through the longitudinal vibration is also minimized.

FIG. 8B illustrates another embodiment of the transmission member 502 having a tapered structure in combination with attenuation tube 604, where the transmission member 502 and attenuation tube 604 is in an oscillated state. The attenuation tube 604 placed in between the step structure 702 and tapered section 704 has a smaller diameter compared to the attenuation tube 604 disclosed in FIG. 8A, thereby moving the location of the contacting diameter 802 away from the node structure 606 and closer to the step structure 702 where the antinode of the longitudinal vibration is located. Therefore, by adjusting the inner diameter of the attenuation tube 604 or the inclination of the tapered section 704, the location of the contacting diameter 802 may be adjusted in accordance to the locations of nodes or antinodes of vertical or transverse vibrations of the ultrasonic vibrations in order to maximize the attenuation efficiency.

FIG. 8C illustrates the transmission member 502 having a tapered section 704 in combination with the attenuation tube 604 having a non-constant inner diameter. In the illustrated embodiment, the attenuation tube 604 has a non-constant inner diameter in the formed of a tapered portion 804 and a portion 806 with a constant diameter. FIG. 8C illustrates an oscillated state and the attenuation tube 604 is placed between the step member 702 and tapered transmission member 704 is moved towards the node position located near the rubber member 606 through the longitudinal vibration (such as at 47 kHz or 55 kHz) during oscillation. The attenuation tube 604 includes a tapered portion 804 that is stopped at the contacting diameter 802 where the outer diameter of the tapered section 704 equals the inner diameter of the tapered portion 804. Including the tapered portion 804 allows the contact area between the transmission member 502 and the attenuation tube 604 to be larger, thereby increasing the attenuation efficiency.

The embodiments disclosed in FIGS. 6, 7 and 8A-C are operable through a configuration in which the node structure 606 is placed towards the distal end of the transmission member 502 and the tapered section 704 and step structure 702 placed towards the proximal end of the transmission member 502 or an opposite configuration in which the node structure 606 is placed towards the proximal end of the transmission member 502 and the tapered section 704 and step structure 702 placed towards the distal end of the transmission member 502.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. A surgical treatment device, comprising:
a transducer generating ultrasonic vibrations;
a transmission rod including a treatment probe, wherein a proximal end of the transmission rod is operatively connected to the transducer for transmitting ultrasonic vibration generated by the transducer to the treatment probe located at a distal end of the transmission rod; and
a damping feature for attenuating vibrations movably placed along the longitudinal axis direction of the transmission rod between an adjacent node and antinode of the longitudinal vibration,
wherein the transmission rod includes a tapered portion, and
wherein the outer surface of the tapered portion has a maximum outer diameter that is larger than an inner diameter of the damping feature.

2. The surgical treatment device according to claim 1, wherein the outer surface of the tapered portion is inclined inwardly from a distal end of the tapered portion towards a proximal end of the tapered portion, and
wherein, along the transmission rod, the node of the longitudinal vibration is located more distally than the tapered portion of the transmission rod.

3. The surgical treatment device according to claim 1, wherein the outer surface of the tapered portion is inclined inwardly from a distal end of the tapered portion towards a proximal end of the tapered portion, and
wherein, along the transmission rod, the node of the longitudinal vibration is located more proximally than the tapered portion of the transmission rod.

4. The surgical treatment device according to claim 1, wherein the damping feature is a tube.

5. The surgical treatment device according to claim 1, wherein the damping feature is a sleeve.

6. The surgical treatment device according to claim 1, wherein the damping feature includes a tapered portion.

7. The surgical treatment device according to claim 1, wherein the damping feature includes a slit.

8. The surgical treatment device according to claim 1, wherein a rubber ring is placed at the node.

9. The surgical treatment device according to claim 1, wherein the transmission rod includes a first portion having an outer diameter larger than the inner diameter of the damping feature, and
wherein, along the transmission rod, the first portion is placed more proximally than the tapered portion of the transmission rod.

10. The surgical treatment device according to claim 9, wherein the first portion is located proximate to the antinode of the longitudinal vibration.

11. The surgical treatment device according to claim 9, wherein the first portion is removable from the transmission rod.

12. The surgical treatment device according to claim 9, wherein the first portion is assembled together with the transmission rod through a screw.

13. The surgical treatment device according to claim 9, wherein the first portion is assembled together with the transmission rod through shrink fitting.

14. The surgical treatment device according to claim 1, wherein the treatment probe is configured to treat living tissue.

15. The surgical treatment device according to claim 1, wherein the treatment probe is configured as an electrode for treatment using high frequency currents.

16. The surgical treatment device according to claim 1, wherein the treatment probe includes a curved shape.

17. The surgical treatment device according to claim 1, wherein the treatment probe includes one or more jaws.

18. A transmission rod, comprising:
an elongate body configured for transmitting ultrasonic vibration from a proximal end to a distal end; and
a treatment probe formed at the distal end of the elongate body, wherein the treatment probe includes a treatment surface,
wherein the transmission rod includes a tapered portion having an outer surface that is inclined inwardly from a distal end of the tapered portion towards a proximal end of the tapered portion,

19. The transmission rod according to claim 18, wherein the transmission rod includes a first portion having an outer diameter larger than the other portions of the transmission rod.

20. The transmission rod according to claim 19, wherein the first portion is removable from the transmission rod.
